(19) United States
(12) Patent Application Publication
(10) Pub. No.: US 2005/0181083 A1
(43) Pub. Date: Aug. 18, 2005

(54) Diet food product

(75) Inventors: Kinya Takagaki, Fukuoka (JP); Takeshi Mitsui, Fukuoka (JP)

Correspondence Address: AMIN & TUROCY, LLP, 1900 EAST 9TH STREET, NATIONAL CITY CENTER, 24TH FLOOR,, CLEVELAND, OH, 44114, US

(73) Assignee: Toyo Shinyaku Co., Ltd.

(21) Appl. No.: 11/020,436

(22) Filed: Dec. 22, 2004

(30) Foreign Application Priority Data
Apr. 18, 2003 (JP) 2003-114710

(51) Int. Cl. A61K 35/78; A61K 31/7048; A61K 31/353

(52) U.S. Cl. 424/770; 514/456; 514/27

(57) ABSTRACT

The present invention provides a diet food product comprising proanthocyanidins (A) and a food material (B) for improving metabolism. This diet food product provides an effect of promoting basal metabolism.

### BACKGROUND OF THE INVENTION

1. Field of the Invention

The present invention relates to a diet food product.

2. Description of the Related Art

In recent years, Japanese eating habits have changed greatly, and Japanese people have come to eat meat-oriented high-fat diets, and furthermore, stress and lack of exercise have become significant. Thus, obesity and associated increase in the body fat percentage have become a problem not only in the middle and old aged persons but also in the young persons.

Most recently, elimination of obesity by reducing body fat through both therapeutic exercise and dietetic therapy has been proposed. Therapeutic exercise is literally a method of taking exercise so as to increase body temperature and increase basal metabolism so that the body uses up more calories, thereby decreasing body fat. However, most people do not have time for such exercise, and it is difficult for them to practice this method. Therefore, dietetic therapy are widely employed, and a variety of types of diet food products are commercially available (e.g., Japanese Laid-Open Patent Publication Nos. 5-284937, 7-147935, and 2000-041628). These diet food products provide a feeling of fullness by eating, while they are low in calories. However, by eating these diet food products, the daily nutrition intake may become insufficient. Moreover, even if the weight is reduced, there is the possibility that the body fat percentage cannot be substantially decreased, and ill health such as malnutrition may be caused. Diet food products for decreasing the body fat percentage also have been proposed (e.g., Japanese Laid-Open Patent Publication Nos. 7-242551, 10-290681, and 11-253130). However, these food products do not have an activity of consuming fat by increasing basal metabolism. Furthermore, during a diet, basal metabolism of the body tends to decrease due to inadequate nutrition, and thus, an efficient diet effect cannot be achieved. Generally, therapeutic exercise and dietetic therapy during a diet cause stress because these methods create a feeling of being restricted, and thus, the weight often increases instead of decreasing. Thus, a food product providing an effect of consuming fat by increasing basal metabolism of the body has not yet been obtained.

Therefore, there is a demand for a food product that increases basal metabolism so that fat can be consumed efficiently without a need for dietary restriction, and thus, makes an efficient diet possible.

### SUMMARY OF THE INVENTION

The inventors of the present invention focused on the facts that body fat can be reduced by increasing basal metabolism of the body and that the basal metabolism can be increased by improving blood circulation, and conducted in-depth research on a food for reducing body fat, that is, a food for dieting successfully. As a result, the inventors of the present invention found a combination of a food material providing an effect of increasing basal metabolism and a substance for helping that effect to be achieved effectively, and thus, achieved the present invention.

A diet food product of the present invention comprises a proanthocyanidin (A) and a food material (B) for improving metabolism, wherein the food material (B) is a food material providing an effect of improving carbohydrate metabolism or a food material providing an effect of improving lipid metabolism.

In a preferred embodiment, the diet food product further comprises a food material (C) providing a sedative effect.

In a preferred embodiment, the diet food product further comprises a food material (D) having a female hormone-like activity.

In a preferred embodiment, the proanthocyanidin (A) is derived from a pine bark.

The diet food product of the present invention comprises proanthocyanidins and a food material for improving metabolism. By ingestion of this food product, carbohydrate metabolism and lipid metabolism are improved, and furthermore, blood circulation is improved, so that fat-burning is promoted, and thus, a diet effect can be achieved. Therefore, there is no need for unreasonable dietary restriction.

### DESCRIPTION OF THE PREFERRED EMBODIMENT

Hereinafter, the diet food product of the present invention will be described. It should be noted that the following description is not limiting the present invention, and it is apparent to those skilled in the art that various alternations can be made within the scope of the spirit of the present invention.

The diet food product of the present invention comprises proanthocyanidins (A) and a food material (B) providing an effect of improving carbohydrate metabolism or an effect of improving lipid metabolism. If necessary, the food product further comprises a food material (C) providing a sedative effect, a food material (D) having a female hormone-like activity, an additive (E), and the like. Hereinafter, these components of the food product will be described.

(A) Proanthocyanidins

In the present invention, proanthocyanidins refer to a group of compounds that are condensation products having flavan-3-ol and/or flavan-3,4-diol as a constituent unit and having a degree of polymerization of 2 or more.

As the proanthocyanidins, proanthocyanidins containing a large amount of condensation products having a low degree of polymerization are preferably used. As such condensation products, condensation products having a degree of polymerization of 2 to 30 (dimer to 30-mer) are preferable, condensation products having a degree of polymerization of 2 to 10 (dimer to decamer) are more preferable, and condensation products having a degree of polymerization of 2 to 4 (dimer to tetramer) are even more preferable. In this specification, the condensation products having a degree of polymerization of 2 to 4 are referred to as oligomeric proanthocyanidins (OPCs). Proanthocyanidins, which are one type of polyphenol, are potent antioxidants produced by plants, and contained concentratedly in portions of plant leaves, bark, or skin or seeds of fruits. More specifically, proanthocyanidins, in particular, OPCs are contained in the bark of pine, oak, bayberry, and the like; the fruit or seeds of grape, blueberry, strawberry, avocado, locust, and cowberry; the hull of barley, wheat, soybean, black soybean, cacao, adzuki bean, conker; the inner skin of peanuts; and the leaves of ginkgo, for example. Moreover, it is known that OPCs are also contained in cola nuts in West Africa; the roots of Rathania in Peru; and Japanese green tea. OPCs cannot be produced in the human body.

The proanthocyanidins have an antioxidation ability and further provide an effect of decreasing cholesterol in blood, an effect of lowering a high blood pressure, an effect of maintaining the elasticity of blood vessels, and an effect of preventing adhesion of cholesterol. Since these effects are achieved synergistically, blood flow in the body is improved, so that blood circulation at the extremities in the body can be increased, and consequently, basal metabolism can be increased. Therefore, fat-burning can be promoted, and thus, a superior diet effect can be achieved.

In particular, when proanthocyanidins having a high OPC content or an extract containing proanthocyanidins having a high OPC content is used, a better diet effect can be achieved than in the case where proanthocyanidins having a high degree of polymerization (i.e., having a low OPC content) are used.

Among the above-described plants containing proanthocyanidins, pine bark contains OPCs abundantly and thus, pine bark can be preferably used as a raw material of the proanthocyanidins.

Hereinafter, a method for preparing an extract containing proanthocyanidins as the main component will be described taking a case in which pine bark that contains OPCs abundantly is used as a raw material plant as an example.

As the pine bark extract, an extract from the bark of a plant belonging to Pinales, such as French maritime pine (Pinus martima), Larix leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada, can be preferably used. Among these, French maritime pine (Pinus martima) bark extract is preferable.

French maritime pine refers to maritime pines that grow in a part of the Atlantic coastal area in southern France. It is known that the bark of this French maritime pine contains proanthocyanidins, organic acids, and other bioactive substances, and proanthocyanidins from the flavonoid family, which are the main component of the French maritime pine bark, have a potent antioxidation effect of removing active oxygen.

The pine bark extract is obtained by extracting the bark of the above-described pines using water or an organic solvent. When water is used, it is preferable to employ warm water or hot water. In order to increase the extraction efficiency, it is preferable to add a salt such as sodium chloride to the water. As the organic solvent that can be employed for extraction, an organic solvent that is acceptable for production of foods or pharmaceuticals can be employed. Examples of such solvent include methanol, ethanol, 1-propanol, 2-propanol, 1-butanol, 2-butanol, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. These water and the organic solvents may be used alone or in combination. In particular, hot water, aqueous ethanol, and aqueous propylene glycol are preferably used.

The method for extracting proanthocyanidins from the pine bark is not particularly limited, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation using an adsorbent or absorbent.

Moreover, it is also possible to perform supercritical fluid extraction in which an entrainer is added. In this method, extraction is performed using an extracting fluid obtained by adding, for example, ethanol, propanol, n-hexane, acetone, toluene, or another aliphatic lower alcohol, aliphatic hydrocarbon, aromatic hydrocarbon, or ketone at about 2 to 20 W/V % to a supercritical fluid, so that the solubility of a target substance to be extracted, such as OPCs and catechins (described later), in the extracting fluid is dramatically increased or the selectivity of separation is enhanced. Thus, a pine bark extract is obtained efficiently.

Since supercritical fluid extraction can be performed at a relatively low temperature, it has the following advantages: it is applicable for extracting substances that deteriorate or decompose at high temperatures; the extracting fluid does not remain; and the extracting fluid can be recovered and recycled, so that a step of removing the extracting fluid and the like can be omitted, and thus, the process can be simplified.

Furthermore, methods other than those mentioned above can be employed for extraction from pine bark, and the examples of which include a batch method using liquid carbon dioxide, a reflux method using liquid carbon dioxide, a reflux method using supercritical carbon dioxide, and the like.

It is also possible to employ a combination of a plurality of extraction processes to perform extraction from the pine bark. By combining a plurality of extraction processes, pine bark extracts with various components can be obtained.

In the present invention, the pine bark extract that contains proanthocyanidins as the main component is specifically prepared using the following method. However, this method is merely an example, and the present invention is not limited to this method.

First, 1 kg of the bark of French maritime pine is immersed in 3 L of a saturated solution of sodium chloride, and extraction is performed for 30 minutes at 100° C. to obtain an extract liquid (extraction step). Then, the extract liquid is filtrated, and the resultant insoluble material is washed with 500 ml of a saturated solution of sodium chloride to obtain a washed liquid (washing step). The extract liquid and the washed liquid are combined to obtain a crude extract liquid of pine bark.

Next, 250 ml of ethyl acetate is added to this crude extract liquid, mixed, and separated to obtain an ethyl acetate layer. This process is repeated five times, and the obtained ethyl acetate layers are combined. The resultant ethyl acetate extract is added directly to 200 g of anhydrous sodium sulfate for drying. Then, this ethyl acetate extract is filtrated, and the filtrated extract is concentrated under a reduced pressure to a volume of 1/5 of the original filtrated extract. The concentrated ethyl acetate extract is poured into 2 L of chloroform and stirred, and the resultant precipitate is recovered by filtration. Subsequently, this precipitate is dissolved in 100 ml of ethyl acetate, and then the resultant solution is added to 1 L of chloroform to form a precipitate. This process is repeated twice, and thus, a washing process is accomplished. With this method, for example, about 5 g of pine bark extract containing at least 20 wt % of OPCs and at least 5 wt % of catechins can be obtained.

It is preferable that an extract derived from a raw material plant such as the above-described pine bark contains at least 40 wt % of proanthocyanidins. Furthermore, the OPC content in this extract derived from a raw material plant is preferably at least 20 wt % and more preferably at least 30 wt %. As such raw material that has a high proanthocyanidin content, a pine bark extract can be preferably used, as described above.

There is no particular limitation regarding the proanthocyanidin content in the diet food product of the present invention. Usually, the proanthocyanidins can be contained in the food product in such an amount that the daily intake amount of the proanthocyanidins is 3 mg to 2000 mg, preferably 5 mg to 1500 mg, and more preferably 100 mg to 1000 mg. For example, when the above-described pine bark extract is used, this extract can be contained in the food product in such a way that the amount of the proanthocyanidins contained in the food product is within the above-described range.

The above-described plant extract contains catechins as well as proanthocyanidins, in particular, OPCs. The term “Catechins” is a general term referring to polyhydroxyflavan-3-ols. As the catechins, for example, (+)-catechin (this is called catechin in a narrow sense), (−)-epicatechin, (+)-gallocatechin, (−)-epigallocatechin, epigallocatechin gallate, epicatechin gallate, and afzelechin are known. From extracts derived from raw material plants such as the above-described pine bark, gallocatechin, afzelechin, 3-galloyl derivatives of (+)-catechin, and 3-galloyl derivatives of gallocatechin are isolated in addition to (+)-catechin mentioned above. Catechins are known to have a cancer inhibiting ability, an arteriosclerosis preventing ability, a lipid metabolism disorder inhibiting ability, a blood pressure elevation inhibiting ability, a platelet aggregation inhibiting ability, an antiallergic ability, an antiviral ability, an antibacterial ability, a dental caries preventing ability, a halitosis preventing ability, an intestinal flora normalization ability, an active oxygen or free radical eliminating ability, an antioxidation effect, and the like. Moreover, catechins are known to have an ability of inhibiting an elevation of blood glucose (antidiabetic ability). Catechins alone have poor solubility in water and exhibit low bioactivity, but in the presence of OPCs, the solubility in water is increased and also catechins are activated. Catechins work effectively when ingested together with OPCs.

It is preferable that catechins are contained in the above-described raw material plant extract in a ratio of 5 wt % or more. More preferably, a formulation is prepared so that it contains this extract containing at least 20 wt % of OPCs and further contains at least 5 wt % of catechins. For example, when the catechin content in the extract is less than 5 wt %, it is possible to add catechins so that the final catechin content becomes at least 5 wt %. It is most preferable to use a pine bark extract containing at least 20 wt % of OPCs and at least 5 wt % of catechins.

(B) Food Materials for Improving Metabolism

The food material for improving metabolism that is contained in the diet food product of the present invention is a food material providing an effect of improving carbohydrate metabolism or a food material providing an effect of improving lipid metabolism. Examples of this food material for improving metabolism include a food material (B1) providing an effect of promoting carbohydrate metabolism or lipid metabolism and a food material (B2) providing an effect of inhibiting carbohydrate and lipid absorption.

Examples of the food material (B1) providing the effect of promoting carbohydrate metabolism or lipid metabolism include food materials derived from plants such as capsicum, Coleus forskohlii, Garcinia cambogia, citrus, paprika, raspberry, and Maca. Furthermore, food materials that contain ubiquinones (e.g., CoQ10), caffeine, soybean peptide, lecithin, vitamin B group, L-carnitine, branched amino acids, germanium containing compounds, molybdenum containing compounds, or the like can be also employed. Examples of the food material (B2) providing the effect of inhibiting carbohydrate and lipid absorption include Gymnema sylvestre, guava leaves, banaba leaves, and sarasia. Among these, food materials derived from capsicum, CoQ10, Coleus forskohlii, citrus, and L-carnitine are preferred. These food materials may be used alone, but it is preferable to use them in combination.

Among the above-mentioned food materials for improving metabolism, the food material (B1) providing the effect of promoting carbohydrate metabolism or lipid metabolism are contained, in particular, in order to achieve a diet effect by accelerating metabolism of carbohydrate or lipids accumulated in the body and thereby reducing body fat. For example, food materials derived from capsicum increase body temperature mainly because capsaicin, which is a pungent principle of capsicum, and its analogous compounds stimulate the sympathetic nerves. Thus, the food materials derived from capsicum increase basal metabolism in the body, and furthermore, promotes degradation of neutral fat, and thus, consume fat. Moreover, they have an ability of degrading glycogen contained in the liver into glucose, so that they can accelerate carbohydrate metabolism. CoQ10 and branched amino acids relate to the TCA cycle for production of energy. In particular, CoQ10 is also a coenzyme that relates to a electron transfer system that is called the respiratory chain, so that it promotes production of ATP and accelerates basal metabolism, and thus, body fat can be reduced. Coleus forskohlii directly activates adenylate cyclase and accelerates lipid metabolism, and thus, provides an effect of reducing body fat. L-carnitine can transport acyl-CoA (i.e., a long chain fatty acid to which coenzyme A (CoA) is bound) from the cytoplasm into the mitochondria in which β-oxidation enzyme is present, so that the fat undergoes oxidation in the mitochondria, and body fat is reduced. In this way, a diet effect can be achieved.

The food materiall (B2) providing the effect of inhibiting carbohydrate and lipid absorption can promote metabolism of lipids accumulated in the body by inhibiting absorption of carbohydrate and lipids.

The above-described food material for improving metabolism is usually contained in the food product of the present invention in a ratio of 0.05 to 10 parts by weight with respect to 1 part by weight of proanthocyanidins (A). More specifically, the food material for improving metabolism can be contained in the food product in such an amount that the daily intake amount of that food material for improving metabolism is preferably 0.003 g to 5 g, more preferably 0.005 g to 2 g, and even more preferably 0.02 g to 1 g. In the case of a food material derived from capsicum, it is preferable that this food material is contained in such an amount that the daily intake amount of total of capsaicin and its analogous compounds, which are pungent ingredients, is 10 mg or more.

(C) Food Materials Providing a Sedative Effect

The food product of the present invention can further contain a food material providing a sedative effect, if necessary. In the present invention, the food material providing a sedative effect refers to a component that can control excitation of the . nerves and the balance of the autonomic nerve system and provides effects of improving neurosis, hypersensitiveness, or insomnia and furthermore, provides an effect of calming the mind. The food material providing a sedative effect also refers to a material containing the above-mentioned component. Examples of such food material include compounds such as hypericin and serotonin and materials containing a component providing the effect mentioned above. Examples of such materials include plants containing the above-mentioned component and extracts from such plants. For example, hop (Humulus lupulus), maypop (Passiflora incarnata), evening primrose (Oenothera biennis), Valeriana officinalis, Leonurus Cardiaca, and Saint John's wort, and their extracts, extract from dioscorea rhizome, and extracts from citrus fruits such as orange and grapefruit can be employed. Preferably, a citrus fruit extract is used.

These food materials providing a sedative effect reduce mental stress during a diet, so that metabolic balance in the body can be stabilized, and also temporal intemperance in eating and drinking, which may occur during a diet, can be inhibited, and thus, a synergistic diet effect can be achieved.

The amount of the food material providing a sedative effect that is contained in the food product of the present invention varies depending on the type of the food material. For example, it is preferable that the food material providing a sedative effect is contained in the food product in such an amount that the daily intake amount is 60 mg to 500 mg and preferably 120 mg to 300 mg in the case of a hop extract, 50 mg to 300 mg in the case of a maypop extract, 80 mg to 900 mg in the case of a Hypericum perforatum extract, 50 mg to 500 mg in the case of a Valeriana officinalis extract, and 5 mg to 300 mg in the case of an orange extract.

(D) Food Materials Having a Female Hormone-Like Activity

The food product of the present invention may further contain a food material having a female hormone-like activity, if necessary. In the present invention, the food material having a female hormone-like activity refers to a component having a female hormone-like activity, such as isoflavone and estrone, and a plant or plant extract containing that component. The food materials having a female hormone-like activity resemble estrogen, a female hormone, in their effect.

Examples of the food material having a female hormone-like activity include soybean, the seeds of pomegranate (Punica granatum), Trifolium pratense, sage (Salvia officinalis), Cimicifuga racemosa, pumpkinseed, and wild yam (Dioscorea villosa), and their extracts. A soybean extract and a Cimicifuga racemosa extract are preferable, and a soybean extract is more preferable.

These food materials having a female hormone-like activity control the hormone balance and prevent chapped skin, reduction in bone density, and weakening of blood vessels due to a diet, and thus, a diet effect can be achieved while reducing the load on the body.

The amount of the food material having a female hormone-like activity contained in the food product of the present invention varies depending on the type of the food material, but it is preferable that the food material is contained in the food product of the present invention in such an amount that the recommended daily intake amount for an adult is satisfied. The recommended daily intake amount for an adult is, for example, about 3 mg to 1000 mg and preferably 5 mg to 500 mg in the case of a soybean extract containing 10 wt % of isoflavone. In the case of a plant extract containing a component having a female hormone-like activity other than isoflavone, usually, it is preferable that the amount of the plant extract that is ingested is in the range of 10 mg to 500 mg. For example, the amount is 30 mg to 300 mg and preferably 80 mg to 200 mg in the case of a Cimicifuga racemosa extract, and 10 mg to 200 mg and preferably 20 mg to 100 mg in the case of a pomegranate seed extract.

(E) Additives

The food product of the present invention further contains a variety of types of additives that are usually used for foods, if necessary. Examples of such additives include nutritional supplements, excipients, extenders, binders, thickeners, emulsifiers, coloring agents, flavors, seasonings, and food additives. For example, it is possible to add royal jelly, vitamins, proteins, lecithin, or the like as a nutritional supplement, and further add a sugar solution or a seasoning agent so as to control taste.

The diet food product of the present invention can be obtained by mixing the proanthocyanidins (A) and the food material (B) for improving metabolism, and furthermore, the food material (C) providing a sedative effect, the food material (D) having a female hormone-like activity, the additives (E) and the like, if necessary, and the resultant mixture can be molded into a predetermined form. For example, the diet food product can be produced in the form of capsules such as hard capsules or soft capsules, tablets, or pills, or it can be produced in the form of powder, granule, candy, or the like. The diet food product may be eaten as it is, or may be dissolved in water, hot water, milk, or the like and drunk.

As described above, the diet food product of the present invention contains proanthocyanidins (A) and a food material (B) for improving metabolism and, preferably, further contains a food material (C) providing a sedative effect and/or a food material (D) having a female hormone-like activity. Thus, the components can exert their specific effects on various factors that cause obesity without offsetting each other, and furthermore, the effect of improving carbohydrate metabolism, the effect of improving lipid metabolism, the effect of improving blood circulation, and the like of these components act synergistically to promote fat-burning, and as a result, an excellent diet effect can be achieved. According to the diet food product of the present invention, there is no need for unreasonable dietary restriction because a diet effect can be achieved by promoting fat-burning.

### EXAMPLES

### Example 1

A pine bark extract (trade name: Flavangenol, produced by TOYO SHINYAKU Co., Ltd.) containing 40 wt % of proanthocyanidins (OPC content: 20 wt % in the extract), a spice extract (Nippon Shinyaku Co., Ltd.) containing 2.7 wt % of capsaicin, crystalline cellulose, sucrose ester, silicon dioxide, and eggshell calcium were mixed in a ratios (wt %) shown in Table 1 below, and tablets (weight of tablets: 200 mg per tablet) were produced from the resultant mixture. The tablets were referred to as “Food 1”.

A total of 30 volunteers who had made an unsuccessful attempt to go on a diet served as subjects. Among them, 10 volunteers who were randomly selected and assigned to one group ingested the above-described Food 1, and the effect of improving basal metabolism and the effect of improving blood circulation were evaluated in the following manner.

First, before the ingestion of the Food 1, skin temperature at the extremities of the above-described 10 volunteers was measured using a thermography (TVS 600, Nippon Avionics Co., Ltd.). The volunteers took a rest for one hour until the measurement, and the measurement was made at three points of the opisthenar of the left hand, i.e., the tip of the middle finger, the midpoint of the proximal phalanx of the middle finger, and the midpoint of the third metacarpal bone. An average temperature of the obtained measurement values was employed as the skin temperature at the extremities. This skin temperature at the extremities was taken as “(I)”. Then, the volunteers soaked their left hand in cold water at 15° C. for 10 seconds, and thus, a cold water load was applied. Subsequently, skin temperature at the extremities was measured 10 minutes after the volunteers pulled out their left hand. This skin temperature was taken as “(II)”.

Then, each of the volunteers ingested 30 tablets of the Food 1 daily for four weeks. The volunteers were only required to keep ingesting the daily requirement (30 tablets) of the Food 1. They were also told that although they ingested the Food 1, they did not have to restrict the foods that they had eaten until then and that there was no particular restriction regarding the form of food ingestion (timing and frequency of ingestion).

After the four-week ingestion, the skin temperature at the extremities and the skin temperature at the extremities after the cold water load were measured in the same manner as described above. These skin temperatures were taken as “(III)”and “(IV)”, respectively.

The difference between the obtained skin temperatures at the extremities before and after the ingestion, that is, (III)-(I), was calculated to evaluate the effect of promoting basal metabolism. Furthermore, the difference between the obtained skin temperatures at the extremities before and after the cold water load that were measured after the ingestion, that is, (IV)-(III), was calculated to evaluate the effect of improving blood circulation. Table 2 shows the results.

### Comparative Examples 1 and 2

In Comparative Example 1, tablets that contain the pine bark extract but do not contain the spice extract were produced according to the process of producing Food 1 in Example 1 using the components shown in Table 1. The resultant tablets were referred to as “Food 2”. In Comparative Example 2, tablets that contain the spice extract but do not contain the pine bark extract were produced according to the process of producing Food 1 in Example 1 using the components shown in Table 1. The resultant tablets were referred to as “Food 3”. The remaining volunteers were randomly divided into two groups of 10 persons each, and the effect of improving basal metabolism and the effect of improving blood circulation were evaluated in the same manner as in Example 1. Table 2 shows the results.

TABLE 1

| | | Ex. 1 | Com. Ex. 1 | Com. Ex. 2 |
|---|---|---|---|---|
| Components | | Food 1 | Food 2 | Food 3 |
| (A) | Pine bark extract | 20 | 20 | — |
| (B) | Spice extract | 20 | — | 20 |
| Additives | Crystalline cellulose | 10 | 10 | 10 |
| | Sucrose ester | 5 | 5 | 5 |
| | Silicon dioxide | 2 | 2 | 2 |
| | Eggshell calcium | 43 | 63 | 63 |

Unit: wt %

TABLE 2

| | | Skin temperature at the extremities (° C.) ^{*1} | | | | | |
|---|---|---|---|---|---|---|---|
| | | Before ingestion of food product | | After ingestion of food product | | Effect of promoting | Effect of improving |
| | | Before cold | 10 min. after | Before cold | 10 mm. after | basal metabolism | blood circulation |
| | Food | water load | cold water load | water load | cold water load | (III)-(I) | (IV)-(III) |
| | ingested | (I) | (II) | (III) | (IV) | (° C.) | (° C.) |
| Ex. 1 | Food 1 | 24.12 ± 0.55 | 26.15 ± 0.78 | 28.21 ± 1.08 | 33.11 ± 1.08 | 4.90 | 4.09 |
| Com. | Food 2 | 23.92 ± 0.54 | 25.89 ± 0.98 | 24.11 ± 0.74 | 27.32 ± 0.76 | 3.21 | 0.19 |
| Ex. 1 | | | | | | | |
| Com. | Food 3 | 23.58 ± 0.61 | 26.28 ± 0.54 | 23.89 ± 1.07 | 27.95 ± 0.99 | 4.06 | 0.31 |
| Ex. 2 | | | | | | | |

^{*1} The values indicate the average value ± standard deviation.

The results in Table 2 show that in the group of volunteers ingesting Food 1, the skin temperature at the extremities that was measured when the volunteers were at rest after the ingestion of the food was higher than that in the group ingesting the Food 2 or the Food 3. Thus, it can be seen that basal metabolism was more promoted (the effect of promoting basal metabolism was obtained) in the group of volunteers ingesting the Food 1 compared with the group of volunteers ingesting the Food 2 or the Food 3. Usually, when body temperature is increased, then basal metabolism is promoted. Such promotion of basal metabolism was seen even at the extremities, and thus, it can be seen that basal metabolism in the entire body was promoted.

Furthermore, recovery of the skin temperature at the extremities after the cold water load was more rapid (i.e., the effect of improving blood circulation was obtained) in the group ingesting the Food 1 than in the groups ingesting the Food 2 or the Food 3. Thus, it can be seen that an excellent effect of improving blood circulation can be achieved by ingestion of the Food 1.

### Example 2

The pine bark extract containing 40 wt % of proanthocyanidins (OPC content: 20 wt % in the extract) in Example 1 and a capsicum extract (Nippon Shinyaku Co., Ltd.) were dissolved in water in the ratios (wt %) shown in Table 3 to obtain a beverage. This beverage was employed to evaluate the diet effect on rats based on the body weight gain inhibition rate in the following manner.

First, male SD rats (Charles River Japan, Inc.) at the age of four weeks were given only a standard feed and water for one week for acclimation. Then, the body weight of each of the rats was measured, and the rats were divided into groups of 5 each so that the average of the weights was almost equal among the groups. Next, each of the rats was orally administered 1 mL of the beverage daily for seven days with a sonde. The rats were allowed to freely ingest drinking water that was a 25% fructose solution and a feed that was the standard feed. A control group in which purified water was administered in place of the above-described beverage in the same manner using a sonde was provided.

On the day 7 from the day when the oral administration started, the body weight and the feed intake amount of each of the rats were measured using a weight scale. Based on the obtained values of the body weight of each of the rats before and after the ingestion of the feed, the body weight gain rate was calculated using the formula (1) below. Furthermore, the obtained body weight gain rate was used to calculate the body weight gain inhibition rate based on the formula (2) below. The weight of the feed before the oral administration and the weight of the feed after the oral administration were measured, and the difference between these weights was employed to obtain the daily average of the feed intake amount. Table 4 shows the results.

$$\begin{matrix}\begin{matrix}Body weight gain \\ rate ( \% )\end{matrix} = \frac{\begin{matrix}\{ ( \begin{matrix}Body weight \\ after ingestion of feed\end{matrix} ) - \\ ( \begin{matrix}Body weight \\ before ingestion of feed\end{matrix} ) \}\end{matrix}}{Body weight before ingestion of feed} \times 100 & Formula ( 1 ) \\ \begin{matrix}Body weight gain \\ inhibition rate ( \% )\end{matrix} = \frac{\begin{matrix}\{ ( \begin{matrix}Body weight gain rate \\ in control group\end{matrix} ) - \\ ( \begin{matrix}Body weight gain rate \\ in test group\end{matrix} ) \}\end{matrix}}{Body weight gain rate in control group} \times 100 & Formula ( 2 )\end{matrix}$$

### Examples 3 to 7

An aqueous solution was prepared using one or two of a Coleus forskohlii extract (Santrex Co., Ltd.), L-carnitine (KONGO CHEMICAL CO., LTD.), CoQ10 (NISSHIN PHARMA INC.), and citrus extract powder (MATSUURA KAMPO KK) as shown in Table 3 in place of the capsicum extract in Example 2 so that the concentrations in each of the components in the aqueous solution were as shown in Table 3. Operations were performed in the same manner as in Example 2 except that this aqueous solution was used as the beverage, and the body weight gain rate, the feed intake amount, and the body weight gain inhibition rate were obtained. Table 4 shows the results.

### Comparative Examples 3 to 9

An aqueous solution was prepared so that it contains the components shown in Table 3 at the concentrations shown in Table 3, and used as a beverage. This beverage was used to perform the same test as in Example 2, and the body weight gain rate, the feed intake amount, and the body weight gain inhibition rate were obtained. Table 4 shows the results.

TABLE 3

| | | | | | | | | Com. | Com. | Com. | Com. | Com. | Com. | Com. |
|---|---|---|---|---|---|---|---|---|---|---|---|---|---|---|
| Components | | Ex. 2 | Ex. 3 | Ex. 4 | Ex. 5 | Ex. 6 | Ex. 7 | Ex. 3 | Ex. 4 | Ex. 5 | Ex. 6 | Ex. 7 | Ex. 8 | Ex. 9 |
| (A) | Pine bark extract | 0.01 | 0.01 | 0.01 | 0.01 | 0.01 | 0.01 | 0.01 | | | | | | |
| (B) | Capsicum extract | 0.01 | | | | | | | 0.02 | | | | | |
| | Coleus forskohlii extract | | 0.02 | | | | 0.01 | | | 0.02 | | | | 0.01 |
| | L-carnitine | | | 0.02 | | | 0.01 | | | | 0.02 | | | 0.01 |
| | CoQ10 | | | | 0.02 | | | | | | | 0.02 | | |
| | Citrus extract powder | | | | | 0.01 | | | | | | | 0.01 | |

Unit: wt %

TABLE 4

| | Body weight | Feed intake | Body weight |
|---|---|---|---|
| | gain rate | amount | gain inhibition |
| | (%) | (g) | rate (%) |
| Example 2 | 38.1 ± 3.1 | 23.4 ± 2.7 | 29% |
| Example 3 | 45.2 ± 2.2 | 24.7 ± 2.5 | 15% |
| Example 4 | 43.9 ± 2.1 | 23.6 ± 3.8 | 18% |
| Example 5 | 44.1 ± 3.1 | 25.0 ± 3.1 | 17% |
| Example 6 | 44.5 ± 2.4 | 23.2 ± 2.8 | 17% |
| Example 7 | 40.2 ± 2.1 | 24.3 ± 3.0 | 25% |
| Com. Ex. 3 | 51.2 ± 3.2 | 23.4 ± 4.0 | 4% |
| Com. Ex. 4 | 47.9 ± 2.7 | 25.1 ± 2.4 | 10% |
| Com. Ex. 5 | 49.1 ± 2.4 | 23.4 ± 1.8 | 8% |
| Com. Ex. 6 | 48.1 ± 2.3 | 24.8 ± 2.2 | 10% |
| Com. Ex. 7 | 50.1 ± 2.9 | 24.9 ± 2.6 | 6% |
| Com. Ex. 8 | 52.1 ± 2.0 | 23.9 ± 3.1 | 2% |
| Com. Ex. 9 | 49.2 ± 3.0 | 24.4 ± 2.3 | 8% |
| Control | 53.4 ± 2.5 | 24.4 ± 3.6 | — |

Referring to the results in Table 4, it can be seen that the beverages of Examples 2 to 7 that contain the pine bark extract and the food material for improving metabolism provide a better diet effect (exhibit a higher body weight gain inhibition rate) than the beverages of Comparative Examples 3 to 9 that contain only one of the pine bark extract and the food material for improving metabolism. Furthermore, among Examples 2 to 7, a particularly high diet effect could be achieved in Example 2 in which the capsicum extract was used as the food material for improving metabolism and in Example 7 in which two food materials for improving metabolism were used. It should be noted that in the examples and comparative examples, there was no particular difference in the feed intake amount, and thus, it was confirmed that the feed intake amount did not affect the body weight gain in all of the groups.

### Example 8

A combination of the pine bark extract and the capsicum extract that exhibited an excellent diet effect in Example 2 was employed. An aqueous solution containing these extracts in the ratio shown in Table 5 was prepared and used as a beverage. Using this beverage, the diet effect and the effect of improving blood circulation on the human body were evaluated as described below.

The diet effect was evaluated in the following manner. First, the body weight and the body fat percentage of ten volunteers were measured using a weight scale equipped with a body fat scale (Koyo Sangyo K.K.). Then, each of the volunteers ingested 30 mL of the above-described beverage three times a day for four weeks, and on the next day after the last ingestion, the body weight and the body fat percentage were measured. The obtained body weight and body fat percentage before the ingestion were taken as “a₁” (kg) and “b₁” (%), respectively, and the body weight and body fat percentage after the ingestion were taken as “a₂” (kg) and “b₂” (%). These values were employed to calculate the rate of decrease in the body weight (%) and the rate of decrease in body fat percentage (%) of each of the volunteers according to the formulae (3) and (4) below, and average values were obtained. Table 6 shows the results. The volunteers were only required to keep ingesting the daily requirement of the beverage. They were also told that although they ingested this beverage, they did not have to restrict the foods that they had eaten until then and that there was no restriction regarding the form of food ingestion (timing and frequency of ingestion):
Rate of decrease in the body weight (%)={(a₁−a₂)/a₁}×100 Formula (3)
Rate of decrease in the body fat percentage (%)={(b₁−b₂)/b₁}×100 Formula (4)

The effect of improving blood circulation was evaluated in the following manner. First, the blood flow rate of each of the above-described 10 volunteers was measured before ingestion of the beverage. Then, the volunteers ingested the beverage in the same manner as described above, and the blood flow rate was measured again after the four-week ingestion. The blood flow rate was obtained by a measurement in a region under the right forearm skin using a rheometer (laser blood perfusion imager PIM II; Perimed AB, Sweden). Table 7 shows the results. Each of the values in the table indicates the average value±standard error, and larger values indicate a higher blood flow rate and a better effect of improving blood circulation.

### Examples 9 to 11

A beverage was prepared using the components shown in Table 5 that exhibited an excellent diet effect in Example 7. Operations were performed in the same manner as in Example 8, and the diet effect and the effect of improving blood circulation were evaluated. Tables 6 and 7 show the results.

### Comparative Examples 10 and 11

A beverage was prepared using the components shown in Table 5. The resultant beverage does not contain the pine bark extract. Operations were performed in the same manner as in Example 8, and the diet effect and the effect of improving blood circulation were evaluated. Tables 6 and 7 show the results.

TABLE 5

| | | | | | | Com. | Com. |
|---|---|---|---|---|---|---|---|
| Components | | Ex. 8 | Ex. 9 | Ex. 10 | Ex. 11 | Ex. 10 | Ex. 11 |
| (A) | Pine bark | 0.5 | 0.5 | 0.5 | 0.5 | | |
| | extract | | | | | | |
| (B) | Capsicum | 1 | | 1 | | 1 | |
| | extract | | | | | | |
| | Coleus | | 0.5 | | 0.5 | | 0.5 |
| | forskohlii | | | | | | |
| | extract | | | | | | |
| | L-carnitine | | 0.5 | | 0.5 | | 0.5 |
| (C) | Orange peel ^{*1} | | | 0.1 | 0.1 | 0.1 | 0.1 |
| (D) | Soybean | | | 0.1 | 0.1 | 0.1 | 0.1 |
| | extract ^{*2} | | | | | | |

Unit: wt %
*¹ Extract from the peel of orange: produced by Nagaoka Perfumery Co., Ltd.
*² Isoflavone content: 10 wt %; produced by Fujicco Co., Ltd.

TABLE 6

| | Rate of decrease | Rate of decrease |
|---|---|---|
| | in the body weight | in the body fat percentage |
| | (%) | (%) |
| Example 8 | 4.5 ± 1.4 | 3.0 ± 0.9 |
| Example 9 | 5.8 ± 1.3 | 2.4 ± 0.7 |
| Example 10 | 7.9 ± 1.8 | 5.6 ± 1.4 |
| Example 11 | 8.2 ± 2.1 | 5.4 ± 1.8 |
| Comparative | 3.3 ± 1.1 | 0.4 ± 1.0 |
| Example 10 | | |
| Comparative | 3.0 ± 1.2 | 0.1 ± 1.1 |
| Example 11 | | |

Referring to the results in Table 6, it can be seen that the beverages of Examples 8 to 11 that contain the pine bark extract containing proanthocyanidins together with a food material for improving metabolism such as capsicum extract, Coleus forskohlii extract, or L-carnitine provide a better diet effect with the body fat being decreased than the beverages of Comparative Examples 10 and 11 that do not contain the pine bark extract. It can be seen that, among the beverages of Examples 8 to 11, especially the beverages of Examples 10 and 11 that further contain orange peel, which is a food material providing a sedative effect, and a soybean extract, which is a food material having a female hormone-like activity, provide a better diet effect.

TABLE 7

| | Blood flow rate | | |
|---|---|---|---|
| | | | After ingestion - |
| | Before ingestion | After ingestion | before ingestion |
| Example 8 | 1.900 ± 0.053 | 1.989 ± 0.044 | 0.089 ± 0.015 |
| Example 9 | 1.895 ± 0.043 | 1.981 ± 0.048 | 0.086 ± 0.022 |
| Example 10 | 1.908 ± 0.039 | 2.002 ± 0.041 | 0.094 ± 0.021 |
| Example 11 | 1.905 ± 0.049 | 1.99 ± 0.043 | 0.085 ± 0.023 |
| Comparative | 1.899 ± 0.041 | 1.968 ± 0.042 | 0.069 ± 0.012 |
| Example 10 | | | |
| Comparative | 1.901 ± 0.044 | 1.923 ± 0.046 | 0.022 ± 0.024 |
| Example 11 | | | |

Average value ± standard deviation

The results in Table 7 show that the beverages of Examples 8 to 11 that contain the pine bark extract containing proanthocyanidins and the food material for improving metabolism (the capsicum extract or a combination of Coleus forskohlii extract and L-carnitine) exhibit a high effect of improving blood circulation. Thus, it can be seen that these beverages provide an excellent diet effect with a synergistic effect with the ability of improving blood circulation.

The invention may be embodied in other forms without departing from the spirit or essential characteristics thereof The embodiments disclosed in this specification are to be considered in all respects as illustrative and not limiting. The scope of the invention is indicated by the appended claims rather than by the foregoing description, and all changes which come within the meaning and range of equivalency of the claims are intended to be embraced therein.

1. A diet food product, comprising a proanthocyanidin (A) and a food material (B) for improving metabolism, wherein the food material (B) is a food material providing an effect of improving carbohydrate metabolism or a food material providing an effect of improving lipid metabolism.

2. The diet food product of claim 1, further comprising a food material (C) providing a sedative effect.

3. The diet food product of claim 1, further comprising a food material (D) having a female hormone-like activity.

4. The diet food product of claim 1, wherein the proanthocyanidin (A) is derived from a pine bark.

5. The diet food product of claim 2, further comprising a food material (D) having a female hormone-like activity.

6. The diet food product of claim 2, wherein the proanthocyanidin (A) is derived from a pine bark.

7. The diet food product of claim 3, wherein the proanthocyanidin (A) is derived from a pine bark.

8. The diet food product of claim 5, wherein the proanthocyanidin (A) is derived from a pine bark.
